(19) United States
(12) Patent Application Publication
(10) Pub. No.: US 2020/0318130 A1
(43) Pub. Date: Oct. 8, 2020

(54) TRANSGENIC PLANTS WITH ENHANCED RESISTANCE TO ABIOTIC STRESS CONDITIONS

(71) Applicant: UNIVERSITY OF PRETORIA, Pretoria (ZA)

(72) Inventors: Donald Arthur COWAN, Pretoria (ZA); Jasmin MERTENS, Pretoria (ZA); Dominique Elizabeth ANDERSON, Cape Town (ZA)

(21) Appl. No.: 16/955,914

(22) PCT Filed: Dec. 19, 2018
(86) PCT No.: PCT/IB2018/060342

§ 371 Date: Jun. 19, 2020

(30) Foreign Application Priority Data
Dec. 19, 2017 (GB) 1721351.3

(51) Int. Cl. C12N 15/82

(52) U.S. Cl. CPC C12N 15/8273

(57) ABSTRACT

The present invention relates to a transgenic plant having increased tolerance to an abiotic stress, including freeze stress, cold stress, heat stress, salt stress, drought stress, osmotic stress, water stress, oxidative stress and/or ionic, wherein the transgenic plant is transformed with a nucleic acid sequence encoding a WHy protein comprising SEQ ID NO:2. The invention further relates to a method for obtaining a transgenic plant having increased tolerance to an abiotic stress, comprising transforming a plant with a nucleic acid sequence encoding the WHy protein. The present invention also relates to vegetative tissue from the transgenic plant of the invention or produced according to the method of the invention and to seeds containing a nucleic acid sequence encoding the WHy protein.

### BACKGROUND OF THE INVENTION

The present invention relates to transgenic plants having increased tolerance to an abiotic stress, including freeze stress, cold stress, heat stress, salt stress, drought stress, osmotic stress, water stress, oxidative stress and/or ionic stress, wherein the transgenic plant is transformed with a nucleic acid sequence encoding a WHy protein comprising SEQ ID NO:2. The present invention also relates to a method for obtaining a transgenic plant which has increased tolerance to an abiotic stress, the method comprising transforming a plant with a nucleic acid sequence encoding the WHy protein. This invention further relates to vegetative tissue from a transgenic plant of the invention or produced according to the method of the invention and to seeds containing a nucleic acid sequence encoding the WHy protein.

The novel bacterial gene, homologous to the Water Hypersensitive (WHy) domain gene, was identified from an Antarctic desert soil metagenomic library. The bacterial WHy gene encodes a 165 amino acid sequence (18.6 kDa) and has high homology to sequences in the Pseudomonas genome. A previous study has demonstrated that recombinant Escherichia coli expressing the novel WHy protein exhibits significant protection against freeze and cold stress. Here the inventors demonstrate that this novel protein domain, when expressed in Arabidopsis thaliana, induces statistically significant protection of the recombinant seeds, seedlings and adult plants against freeze, cold, desiccation stress, salinity and heat. These findings are of considerable significance in that the inventors have demonstrated a multiple stress-protection phenotype from the expression of a single recombinant protein.

All organisms may face various abiotic stress factors, such as drought, heat, cold, freezing or high salinity, which lead to the multiple deleterious effects including the loss of intracellular water i.e. dehydration. Water deficiency is considered to be the most life threatening abiotic stress as water maintains the structural order of cells, stabilizes proteins, lipids and nucleic acids and maintains a cellular microenvironment in which vital metabolic systems and chemical reactions are possible. In plants, low water potential and related stresses are ‘managed’ by the uptake and loss of water in the cells, accumulation of solutes or the use of protective proteins; all mechanisms aimed at preventing or repairing damage to cellular constituents in order to either avoid or tolerate low water potential.

Large accumulation of very hydrophilic proteins, collectively known as late embryogenesis abundant (LEA) proteins, occurs during the last stages of seed maturation and during plant water deficit and has been described as the common protective mechanism in vegetative organisms under water stress. LEA proteins are part of a more widespread group of proteins (hydrophilins)—which are involved in the physiology of adaption to water deficit and are present in plants, bacteria, yeast and vertebrates. These proteins possess multifunctional capacities to ‘manage’ dehydration and typically show considerable structural plasticity.

The generally accepted classification of the LEA proteins is based on their structural features. A well -characterized group of the LEA proteins is LEA 2, sometimes referred to LEA 14. Dehydration proteins (dehydrins) are important members of the LEA 2 protein family. The architecture of these dehydrins is defined by the presence of three types of conserved sequence motifs; named K-, Y- and S-segments. The K-segment, consisting of a highly conserved 15 amino acid motif, may be especially important as it has been found in all dehydrins.

The functional roles of dehydrins include antioxidant activity, membrane stabilization and intracellular accumulation (‘space filling’) during water stress. Dehydrin gene expression is up-regulated by a wide range of abiotic stress factors, such as drought, cold and salinity.

The water hypersensitive (WHy) domain is a unique component of the LEA 2, Hin1 and LEA 8 protein families and occurs widely in plants, nematodes, bacteria and archaea. The WHy domain is an element of the LEA 2 superfamily, and is typically about 100 amino acids in size in bacteria. Larger protein sequences, of up to 615 aa with multiple WHy domains, have been found in both plants and archaea.

Due to this complex structural architecture, the exact mechanism of the protective physiology of LEA 2 proteins is still not well understood. It has been hypothesized that WHy domain-containing proteins act as stabilizers for membrane-bound proteins during water stress, either by direct interaction with water and small polar molecules, or by binding to the protein surface and replacing the water.

The evolutionary origins of the WHy domain remain unclear. Strong evidence for an early evolutionary appearance in plants, followed by a horizontal transfer from plants to prokaryotes, (i.e. bacteria and archaea) has been presented. Homology with sequences found in the ancient green alga Chlamydomonas reinhardtii (Genbank Accession number: AV395132), as well as the relatively narrow phylogenetic distribution of the WHy domain in bacterial taxa (largely restricted to plant pathogens and symbionts), is thought to support a plant evolutionary origin. More recently, extensive phylogenetic sequence analyses have demonstrated a likely phylogenetic relationship with an ancestral WHy domain in archaea, with a more recent expansion into the plant and bacterial kingdoms.

The inventors have recently described a novel gene, found in an Antarctic desert soil metagenomic library, that codes for a protein homologous to a water hypersensitivity domain (WHy) in Pseudomonas (Anderson et al., 2015). This 165 amino acid 18.6 kDa WHy domain has a largely variant NPN signature motif at the N-terminus (Anderson et al., 2015). Functional studies in E. coli recombinants expressing the WHy domain, both with and without the N-terminal signal sequence, showed active protection of the host cells against cold (growth at +8° C.) and freeze-thaw cycling effects, in vivo (Anderson et al., 2015).

### SUMMARY OF THE INVENTION

The present invention relates to transgenic plants having increased tolerance to an abiotic stress, compared to a control plant, and methods for obtaining such a plant.

In a first aspect of the invention there is provided for a transgenic plant having increased tolerance to an abiotic stress, wherein the transgenic plant is transformed with a nucleic acid sequence encoding a WHy protein comprising SEQ ID NO:2, when compared to a control plant not transformed with a nucleic acid sequence encoding the WHy protein.

In a first embodiment of the invention the nucleic acid sequence encoding the WHy protein is operably linked to a promoter. It will be appreciated by those of skill in the art that the promoter may be either a constitutive promoter, a stress inducible promoter which is induced by an abiotic stress or a tissue-specific promoter.

In a second embodiment of the invention the abiotic stress may be selected from the group consisting of freeze stress, cold stress, heat stress, salt stress, drought stress, osmotic stress, water stress, oxidative stress and/or ionic stress.

In a third embodiment the increased tolerance to the abiotic stress is selected from the group consisting of increased germination efficiency, increased seedling survival, longer root length, faster plant development, increased seedling wet weight, increased plant growth, and early flowering.

In a further embodiment the transgenic plant is transiently or stably transformed with the nucleic acid encoding the WHy protein. Preferably the transgenic plant is stably transformed with the nucleic acid encoding the WHy protein.

In yet a further embodiment, the nucleic acid sequence used to transform the plant is a synthetic codon-optimised sequence for expression in the plant.

In a second aspect of the invention there is provided a method for obtaining a transgenic plant having increased tolerance to an abiotic stress, comprising transforming a plant with a nucleic acid sequence encoding a WHy protein comprising SEQ ID NO:2.

In one embodiment the plant may be a monocotyledonous or dicotyledonous plant. Preferably, the plant is selected from the group consisting of alfalfa, apple, arrowroot, artichoke, avocado, banana, barley, beans, beetroot, blackberry, blueberry, brassicas, broccoli, brussel sprouts, cabbage, canola, cantaloupe, carrot, cassava, cauliflower, cherry, cocoa, coffee, cotton, cucumber, currant, eggplant, endive, grapefruit, grapes, honeydew, kohlrabi, leek, lemons, lettuce, mango, maize, melon, millet, mint, miscane, Miscanthus, oats, oilseed rape, onion, oranges, papaya, peach, peanut, pear, peas, peppers, pineapple, plum, pumpkin, potato, radish, rape, raspberry, rice, rye, setaria, sorghum, soybean, spinach, squash, strawberry, sugarcane, sunflower, sweet potato, switchgrass, wheat, tangerines, teosinte, tobacco, tomato, tomatillo, turfgrass, turnip, and watermelon.

In a first embodiment of the invention, the method further comprises exposing the transgenic plant to an abiotic stress and selecting at least one plant having increased tolerance to the abiotic stress.

In a second embodiment of the invention the nucleic acid sequence encoding the WHy protein is operably linked to a promoter. It will be appreciated by those of skill in the art that the promoter may be either a constitutive promoter, a stress inducible promoter which is induced by an abiotic stress or a tissue-specific promoter.

In a third embodiment of the invention the abiotic stress may be selected from the group consisting of freeze stress, cold stress, heat stress, salt stress, drought stress, osmotic stress, water stress, oxidative stress, and ionic stress.

In a fourth embodiment the increased tolerance to the abiotic stress is selected from the group consisting of increased germination efficiency, increased seedling survival, longer root length, faster plant development, increased seedling wet weight, increased plant growth, and early flowering.

In yet a further embodiment the transgenic plant is transiently or stably transformed with the nucleic acid encoding the WHy protein. Preferably the transgenic plant is stably transformed with the nucleic acid encoding the WHy protein.

In yet a further embodiment, the nucleic acid sequence used to transform the plant is a synthetic codon-optimised sequence for expression in the plant.

In one embodiment the plant may be a monocotyledonous or dicotyledonous plant. Preferably, the plant is selected from the group consisting of alfalfa, apple, arrowroot, artichoke, avocado, banana, barley, beans, beetroot, blackberry, blueberry, brassicas, broccoli, brussel sprouts, cabbage, canola, cantaloupe, carrot, cassava, cauliflower, cherry, cocoa, coffee, cotton, cucumber, currant, eggplant, endive, grapefruit, grapes, honeydew, kohlrabi, leek, lemons, lettuce, mango, maize, melon, millet, mint, miscane, Miscanthus, oats, oilseed rape, onion, oranges, papaya, peach, peanut, pear, peas, peppers, pineapple, plum, pumpkin, potato, radish, rape, raspberry, rice, rye, setaria, sorghum, soybean, spinach, squash, strawberry, sugarcane, sunflower, sweet potato, switchgrass, wheat, tangerines, teosinte, tobacco, tomato, tomatillo, turfgrass, turnip, and watermelon.

In a third aspect of the invention there is provided for vegetative tissue obtained from the transgenic plant described in the first aspect of the invention or the transgenic plant obtained by the method described in the second aspect of the invention.

In a further aspect of the invention there is provided for a transgenic seed containing a nucleic acid sequence encoding a WHy protein comprising SEQ ID NO:2.

### BRIEF DESCRIPTION OF THE FIGURES

Non-limiting embodiments of the invention will now be described by way of example only and with reference to the following figures:

FIG. 1: WHy/ΔWHy PCR with gDNA of T0 and T1 Arabidopsis thaliana generation. (A) WHy PCR using gDNA from 4 different Arabidopsis thaliana WHy recombinants. (B) ΔWHy PCR of 4 different ΔWHy recombinants. Water and genomic DNA from wild-type (WT) Arabidopsis thaliana were used as controls.

FIG. 2: WHy/ΔWHy PCR with cDNA of T0 and T1 Arabidopsis thaliana generation. WHy and ΔWHy PCR was performed using cDNA from 2 different Arabidopsis thaliana WHy and ΔWHy recombinants respectively. Water and genomic DNA from WT Arabidopsis thaliana was used as controls.

FIG. 3: WHy/ΔWHy Western Blot with protein of T0 and T1 Arabidopsis thaliana generation. (A) WHy protein expression in T0 Arabidopsis thaliana. (B) Protein expression in the T1 generation. WHy protein expressed in E. coli and protein from WT plants were used as controls.

FIG. 4: Germination ability of WT and transgenic Arabidopsis thaliana seeds after freeze-shock: (A) Germination of seeds and seedling development for WT, ΔWHy and WHy seeds on MS basal media plates. Transgenic seeds showed strong tolerance to freeze and ability to germinate after freeze treatment while WT seeds showed nearly no tolerance to freezing. (B) Comparison of different seedling development after freeze-shock on seeds. Without freezing there was no significant difference observed between WT and transgenic seeds. But applied freezing on the seeds showed dramatic inhibition of seed germination in WT but not in transgenic lines.

FIG. 5: Germination ability of WT and transgenic Arabidopsis thaliana seeds after freeze-shock: Statistical highly significant differences in ability to germinate after freezing of WT and transgenic seeds. Statistics were done using GraphPad software and unpaired t test.

FIG. 6: Heat stress on Arabidopsis thaliana seeds: Statistically significant differences were observed in ability to germinate after heat-shock on WT and transgenic seeds. Statistics were performed using GraphPad software and unpaired t test.

FIG. 7: Seedling development under long periods of cold temperatures: Plant and root development of seedlings is shown for 2 months of growth at +4° C. Left panel, WT seedlings; Central panel, ΔWHy seedlings; Right panel, WHy seedlings.

FIG. 8: Plant and root development of WT and recombinant Arabidopsis thaliana seedlings during long cold periods: Statistically significant differences were observed in (A) plant root length and (B) wet weight of transgenic seedlings compared to WT plants. Statistics were done using GraphPad software and unpaired t test.

FIG. 9: Freeze treatment on adult Arabidopsis thaliana WT and recombinant plants: 4 weeks old plants were exposed to 5 hours of −5° C. in darkness. Each row shows one example of WT, ΔWHy and WHy Arabidopsis thaliana plants: (A) plants before freeze exposure; (B) immediately after freeze treatment; and (C) the same plants after a 7 day period of recovery under standard conditions.

FIG. 10: Germination of Arabidopsis thaliana seeds on salinity media: Statistically relevant differences were observed in the ability to germinate on 50 mM NaCl in transgenic seeds compared to WT. Statistics were done using GraphPad software and unpaired t test.

FIG. 11: Germination of Arabidopsis thaliana seeds on 100 mM mannitol media: Statistically significant differences were observed in ability to germinate under low-water activity stress in transgenic seeds compared to WT seeds. Statistics were done using GraphPad software and unpaired t test.

FIG. 12: Effect of drought on adult Arabidopsis thaliana WT and recombinant plants: 4 weeks old plants were exposed to a 1 week period of drought. (A) 5 plants each of WHy and ΔWHy recombinant plants before drought; (B) directly after drought; (C) after 1 week recovery; (D) 5 WT plants before drought; and (E) directly after drought.

FIG. 13: Wild type nucleotide sequence of the WHy gene (SEQ ID NO:1).

FIG. 14: Wild type amino acid sequence of the WHy protein (SEQ ID NO:2).

FIG. 15: Arabidopsis thaliana codon-optimised nucleotide sequence of the WHy gene (SEQ ID NO:3).

FIG. 16: Arabidopsis thaliana codon-optimised truncated nucleotide sequence of the WHy gene (SEQ ID NO:5).

FIG. 17: Truncated amino acid sequence of the WHy protein (SEQ ID NO:6).

FIG. 18: Effect of Drought on adult Arabidopsis thaliana WT and recombinant plants: a) Left panel shows no WHy gene expression in seeds while the right panel shows moderate WHy expression on both transgenic lines but not in WT roots. b) Left and right panels exhibit clear WHy expression in all transgenic but not in WT lines.

FIG. 19: WHy expression before and during abiotic stress conditions: A) Upper and lower panel show WHy expression in transgenic lines before and directly after freeze shock and no WHy expression in WT. B) Both upper and lower panels show WHy gene expression before and directly after drought stress (no gene expression in WT) and C) WHy gene expression in both transgenic lines but not in WT plants after development under long periods of cold at +4° C.

### SEQUENCE LISTING

The nucleic acid and amino acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:

SEQ ID NO:1—Wild type nucleotide sequence of the WHy gene.

SEQ ID NO:2—Wild type amino acid sequence of the WHy protein.

SEQ ID NO:3—Arabidopsis thaliana codon-optimised nucleotide sequence of the WHy gene.

SEQ ID NO:4—Histidine tag amino acid sequence.

SEQ ID NO:5—Arabidopsis thaliana codon-optimised truncated nucleotide sequence of the WHy gene.

SEQ ID NO:6—Truncated amino acid sequence of the WHy protein.

SEQ ID NO:7—WHy forward primer nucleotide sequence.

SEQ ID NO:8—WHy forward primer nucleotide sequence.

SEQ ID NO:9—WHy reverse primer nucleotide sequence.

SEQ ID NO:10—Nicotiana benthamiana codon-optimised truncated nucleotide sequence of the WHy gene.

SEQ ID NO:11—Kozak sequence.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

This invention relates to the production of a transgenic plant incorporating a bacterial WHy gene identified from an Antarctic desert soil metagenomic library and which is homologous to Water Hypersensitive domain (WHy) which is a typical component of Late Embryogenesis Abundant (LEA) proteins. The WHy protein is a short 165 amino acid of 18.6 kDa. Recombinant Arabidopsis thaliana plants containing the WHy gene optimized for expression in Arabidopsis thaliana show statistically significant protection of the recombinant seeds, seedlings and adult plants against freeze, cold, desiccation stress, salinity and heat. This invention thus relates to expression of a single recombinant protein in a plant to induce a multiple stress-protection phenotype in the plant.

The terms “nucleic acid”, “nucleic acid molecule” or “polynucleotide” encompass both ribonucleotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogues or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides.

The term “WHy gene” or “WHy” refers to a polynucleotide sequence, of any length, that encodes the WHy protein. The WHy gene is heterologous with respect to the host plant cell. The selected sequence can be a full length or a truncated gene, a fusion or tagged gene and can be a cDNA, a genomic DNA, or a DNA fragment, preferably, a cDNA.

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation).

A “host cell” refers to a cell into which the WHy gene is introduced. The term host cell includes both prokaryotic cells used for propagation of the construct to prepare vector stocks, and eukaryotic cells for expression of the WHy polypeptides of interest, such as plant cells.

The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the activity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as at least about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

In some embodiments, the nucleic acid molecules of the invention are operably linked to other sequences. By “operably linked” is meant that the nucleic acid molecules encoding the WHy polypeptides described herein and regulatory sequences are connected in such a way as to permit expression of the proteins of interest when the appropriate molecules are bound to the regulatory sequences. Such operably linked sequences may be contained in vectors or expression constructs which can be transformed or transfected into host cells for expression. “Regulatory sequences” include but are not limited to promoters, transcription termination sequences, enhancers, splice acceptors, donor sequences, introns, ribosome binding sequences, poly(A) addition sequences, and/or origins of replication.

The term “recombinant” means that something has been recombined. When used with reference to a nucleic acid construct the term refers to a molecule that comprises nucleic acid sequences that are joined together or produced by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e. by human intervention. Recombinant nucleic acid constructs may be introduced into a host cell by transformation.

The term “vector” refers to a means by which polynucleotides or gene sequences can be introduced into a cell. There are various types of vectors known in the art including plasmids, viruses, bacteriophages and cosmids. Generally polynucleotides or gene sequences are introduced into a vector by means of a cassette. The term “cassette” refers to a polynucleotide or gene sequence or gene sequences that is/are expressed from a vector, for example, the polynucleotide or gene sequence encoding the WHy polypeptides.

The following examples are offered by way of illustration and not by way of limitation.

### EXAMPLE 1

Generation of Transgenic Arabidopsis thaliana Plants

Sub-Cloning of WHy and ΔWHy

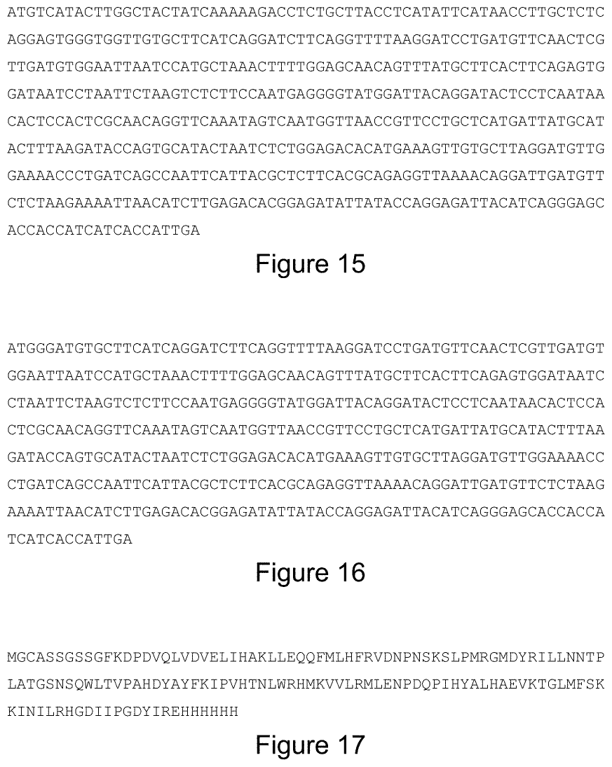

The full-length sequence of the WHy gene (SEQ ID NO:1, FIG. 13) encoding the WHy protein (SEQ ID NO:2, FIG. 14) was codon-optimized and synthesized by GenScript USA Inc. for heterologous expression in Arabidopsis thaliana (SEQ ID NO:3, FIG. 15). The gene was designed to introduce a 6× His Tag (HHHHHH; SEQ ID NO:4) at the C-terminal domain of the protein, to facilitate the detection of the protein after expression. A codon-optimized DNA sequence (SEQ ID NO:5, FIG. 16) of a truncated variant of the WHy protein lacking the predicted signal peptide (SEQ ID NO:6, FIG. 17) was also synthesised. The truncated variant of the WHy protein is referred to herein as ΔWhy and the truncated variant of the WHy gene is referred to herein as ΔWhy. The complete DNA sequences of the optimized gene and of the truncated variant were amplified using the following primers:

```
WHy [forward]
SEQ ID NO: 7
  5′-GCCACCATGGGATACTTGGCTACTATC-3′
ΔWHy [forward]
SEQ ID NO: 8
  5′-GCCACCATGGGATGTGCTTCATCAG-3′
WHy [reverse]
SEQ ID NO: 9
  5′-TCAATGGTGATGATGGTGGTGCTCC-3′
```

Both forward primers were designed to introduce a Kozak sequence (SEQ ID NO:11) at the 5′ end of the insert sequence. Both fragments were cloned in the pMDC32 using the Gateway® Cloning Vector kit (Invitrogen) for constitutive ectopic protein expression in Arabidopsis thaliana.

Transformation of Arabidopsis thaliana

Agrobacterium-mediated transformation of Arabidopsis thaliana was performed via the floral dipping transformation technique (Zhang et al., 2006). Agrobacterium-mediated transformation of Arabidopsis thaliana used the floral dip method (doi:10.1038/nprot.2006.97): i.e., developing Arabidopsis thaliana inflorescences were dipped for a few seconds into a 5% sucrose solution containing 0.01-0.05% (vol/vol) Silwet L-77 and resuspended Agrobacterium cells carrying pMDC32 plasmids with WHy or ΔWHy gene inserts. Treated plants are allowed to set seed and then plated onto a selective medium to screen for recombinants. Transgenic plants were selected by planting seeds on plant nutrient agar plates supplemented with 10 mg/l hygromycin. Resistant seedlings were identified by their long hypocotyls (0.8-1.0 cm): c.f. non-resistant seedlings which had short hypocotyls (0.2-0.4 cm). The insertion of the WHy genes was confirmed by PCR.

Plant Material and Growth Conditions

Columbia-0 (Col-0) ecotype of Arabidopsis thaliana was used for this study. For growth of Arabidopsis thaliana on plates, seeds were surface-sterilized, then plating onto 1× MS basal media with sucrose (0.5% w/v), agar (0.8% w/v), pH 5.0 followed by three days of stratification at +4° C./in darkness. Seeds were incubated in a growth chamber for two weeks at +22° C. under long day conditions (16 hours light, 8 hours dark) with a light intensity of 120-130 μmol m⁻²s⁻¹, and then grown in peat moss bags (Jiffy Products International AS, Norway) under the same conditions.

Identification of the WHy Gene Sequence

Successful cloning and integration of the WHy gene into the genome of Arabidopsis thaliana plants was demonstrated by WHy-PCR with plant DNA as template. Leaves were harvested, quick-frozen in liquid N₂ and stored at −80° C. Genomic DNA was isolated using the NucleoSpin Plant II kit (Macherey-Nagel, Germany) following the suppliers instructions. WHy gene-specific primers were designed for two different primers sets, each flanking the entire gene, where one primer contains the sequence for a potential signal peptide. Primer sequences used were as described above (SEQ ID NOs:7-9). PCRs were performed in T100 thermal cyclers (BIO-RAD, USA) and visualized using 0.8-1% agarose gels electrophoresed for 30-50 min.

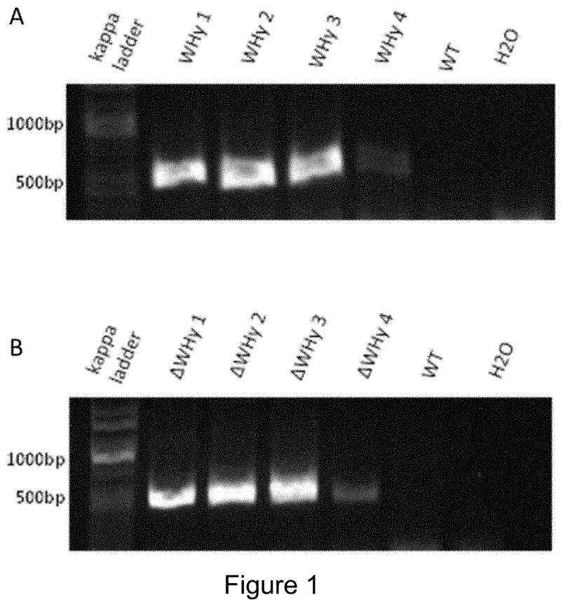

After optimization of the WHy gene sequence, the design of two different gene constructs and the Agrobacterium-mediated transformation of the Arabidopsis thaliana host plant, the successful cloning, integration and stable expression of the WHy gene in the Arabidopsis thaliana genome was demonstrated by PCR analysis of cell extracts for all first and second generation transgenic (T0 and T1) plants. PCR reactions showed a ca. 516 bp product using WHy specific primers and a 485 bp product for ΔWHy PCR, while WT was taken as a negative control with no PCR product observed (FIG. 1). Both T0 and T1 generation plants showed stable integration and inheritance of the recombinant genes in the plant genome.

### EXAMPLE 2

### Expression of the WHy Gene in Transgenic Plants

Gene expression was demonstrated by RT-PCR of the WHy gene. Plant RNA was extracted from quick-frozen Arabidopsis thaliana leaves using the NucleoSpin RNA Plant kit (Macherey-Nagel, Germany) following the suppliers instructions. RT-PCR was performed using the OneStep RT-PCR kit (QIAGEN, Netherlands) following the suppliers instructions, in T100 thermal cyclers (BIO-RAD, USA). PCR products were visualized in 0.8-1% agarose gels electrophoresed for 30-50 min.

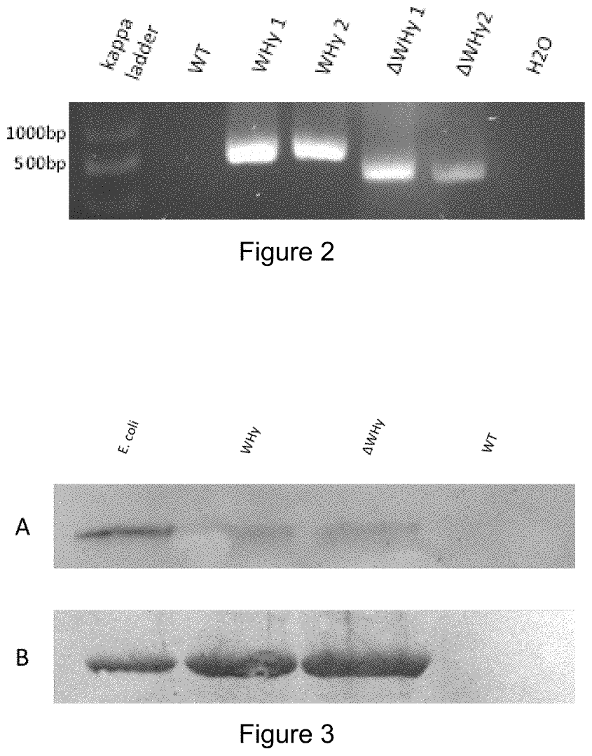

Constitutive expression of the WHy gene, in the absence of any imposed stress treatment, was demonstrated in all recombinant T0 and T1 plants using a one-step RT-PCR reaction (transcribing RNA to cDNA and producing WHy gene amplicon products in a single PCR reaction) (FIG. 2). WT transcripts showed no WHy PCR product (negative control) (FIG. 2).

Identification of the WHy Protein

The successful expression of the WHy protein was demonstrated by Western Blotting. The WHy gene was pre-designed with a His-tag, detectable with Anti-His antibodies (KPL, USA). Total proteins were isolated (using a modified standard protocol: Laing and Christeller, 2004) from transformed Arabidopsis thaliana plants and the recombinant proteins recovered using His-Nickel affinity adsorption. His-tagged protein was visualized by binding with Anti-His-specific Nickel-antibody linked to a reporter system using the HisDetector Western Blot Kit and HisDetector Nickel Conjugates (KPL, USA).

WHy protein expression was demonstrated using Western Blotting for all tested transgenic T0 and T1 generation Arabidopsis thaliana plants. Under standard (non-stressed) growth conditions, moderate WHy and WHy protein expression was detected in all transgenic lines, respectively, while no transgenic protein was observed in WT plants. The E. coli expressed and purified WHy protein (18.6 kDa) was taken as a positive control (FIG. 3).

### EXAMPLE 3

Evaluation of Stress Tolerance of Transgenic Arabidopsis thaliana Plants

Effect of Freeze Stress on Transgenic Arabidopsis thaliana Seed Germination

To monitor the germination efficiency after freezing, WT and WHy/ΔWHy transgenic Arabidopsis thaliana seeds were exposed at −20° C. or −80° C. for 20 min followed by a 20 min recovery at +22° C. (protocols adapted from Xin et al. (1998) and Anderson at al. (2015), respectively). As controls, WT and transgenic seeds were keep at +4° C. (normal storage conditions) for 20 min, followed by a 20 min recovery at +22° C. All temperature treatments were performed in darkness. After surface sterilization (10% bleach and 0.1% Triton X), seeds were generated on MS basal media plates in a controlled culture room set at +22° C. under long day (LD) conditions (16 hours light/8 hours dark) for 14-21 days. Germination efficiency was measured after 3 weeks. Experiments were performed in triplicate.

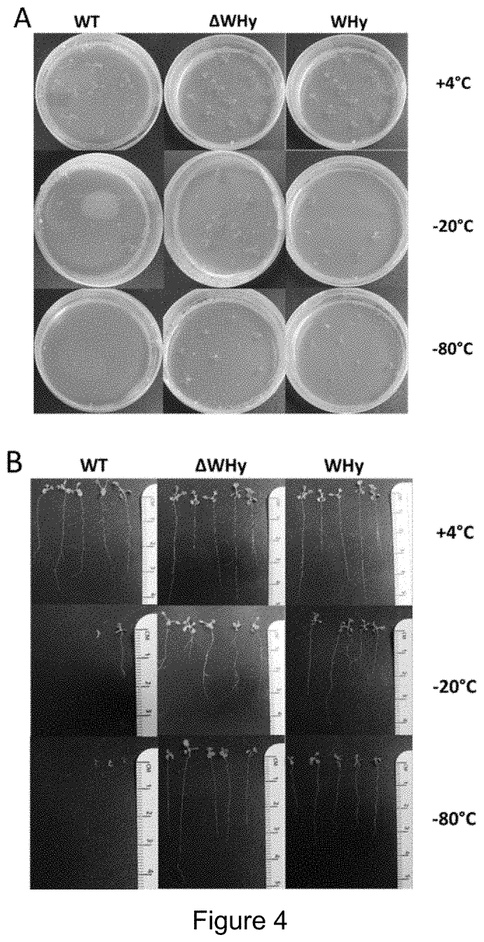

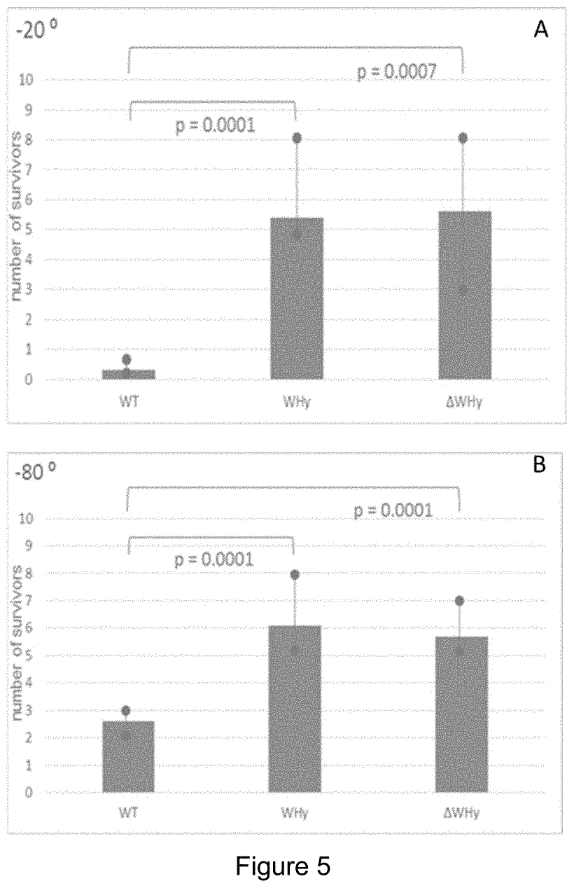

Phenotypic and physiological differences were observed in all transgenic plant lines (WHy and ΔWHy; compared to WT seeds) with respect to germination efficiency and seedling survival after exposing the seeds to temperatures of −20° C. or −80° C. Higher germination efficiency, longer root length and a generally faster development was observed in recombinant plant lines compared to WT plants after freeze-shock and a further growth period of 3 weeks at +22° C. (FIG. 4). While germination of WT and WHy-expressing plants showed no statistically significant difference in germination (p>0.05) without freeze-shock, a dramatic increase (p<0.05) in seedling development was observed only in transgenic lines after freezing treatment (FIG. 5). Germination percentage was calculated as number of seeds germinated per 10 seeds (Table 1). For WT seeds, the percentage germination after freeze-shock ranged from 3 to 16%, while WHy recombinants showed 50 to 66% percent germination, c.f. 36-73% germination for ΔWHy seeds.

Effect of Heat Stress on Transgenic Arabidopsis thaliana Seed Germination

Triplicate sets of 10 seeds (each of WT and transgenic plant lines) were heat stressed at +45° C. for 5 hours. Controls (WT and transgenic seeds) were incubated at +4° C. for 5 hours (normal storage conditions). All temperature treatments were performed in darkness. Germination was performed on MS basal media plates in a controlled culture room at +22° C. under LD conditions for 2-3 weeks. The number of survivors/seedlings was determined and germination percentage calculated.

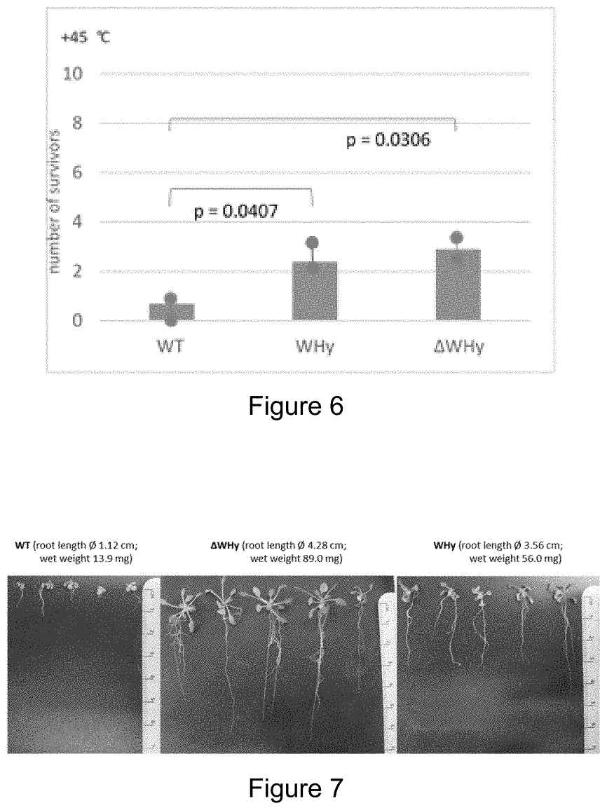

Quantitative assessment of Arabidopsis thaliana seed germination after heat-stress showed that WT seeds exhibited a higher and statistically significant (p<0.05) sensitivity to high temperatures c.f. the recombinants (FIG. 6). Seeds carrying the WHy and ΔWHy genes showed between 2.5- and 6-fold higher germination rates after heat treatment than seeds without the WHy gene (Table 1). Both WT and transgenic lines germinated equally well under standard growth conditions (data not shown).

TABLE 1
Effect of freeze- and heat- shock on recombinant seeds

| | Freeze-shock | | | | Heat-shock | |
|---|---|---|---|---|---|---|
| | −20° C. | | −80° C. | | +45° C. | |
| Line | # survivors/survival % | | # survivors/survival % | | # survivors/survival % | |
| WT | 0.3 + 0.6 | 3.3 | 1.7 + 1.5 | 16.7 | 0.7 + 1.2 | 6.7 |
| WHy 1 | 6.0 + 1.7 | 66.7 | 6.7 + 1.2 | 66.7 | 1.7 + 0.6 | 16.7 |
| WHy 2 | 5.0 + 0.0 | 50.0 | 5.7 + 0.6 | 56.7 | 3.3 + 1.6 | 33.3 |
| WHy 3 | 5.7 + 1.2 | 56.7 | 6.0 + 0.0 | 60.0 | 1.7 + 0.6 | 16.7 |
| ΔWHy 1 | 6.0 + 1.0 | 60.0 | 4.7 + 1.5 | 46.7 | 3.0 + 0.0 | 30.0 |
| ΔWHy 2 | 3.7 + 1.2 | 36.7 | 5.3 + 0.6 | 53.3 | 4.0 + 1.0 | 40.0 |
| ΔWHy 3 | 7.3 + 0.6 | 73.3 | 6.3 + 0.6 | 63.3 | 1.7 + 1.5 | 16.7 |

Effect of Long-Term Cold Treatment in Arabidopsis thaliana Plant Growth

For long-term cold tolerance of seedlings, triplicate sets of 10 seeds (of WT and both transgenic Arabidopsis thaliana lines) were grown for 7 days on MS basal media plates in a controlled culture room at +22° C. and +4° C. for 2 months, both under LD conditions.

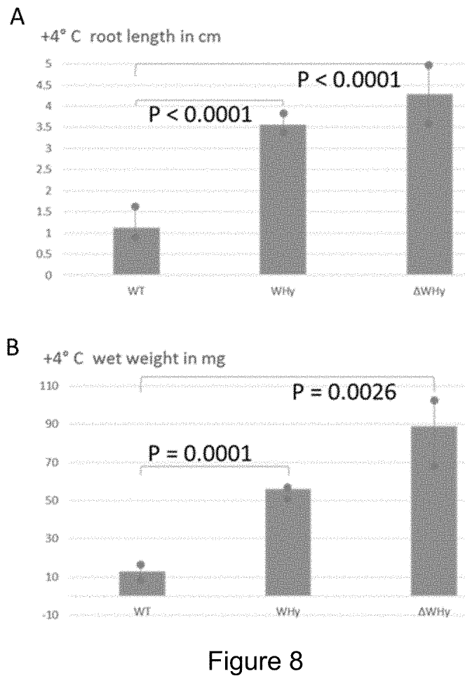

To measure the long-term effects of cold on germination and growth, WT and transgenic Arabidopsis thaliana seeds were exposed to normal germination and growth conditions for 1 week and then grown for 2 month at +4° C. with normal day/night conditions. A dramatic difference was observed between WT and recombinants with respect to root length and wet weight of the seedlings (FIG. 7). Both parameters showed statistically significant increases in tolerance to extended cold periods in transgenic plant lines (FIG. 8).

Effect of Freeze-Shock on Arabidopsis thaliana Survival and Development

Freeze-shock resistance was determined on 4 weeks old adult plants grown under normal conditions (+22° C./LD). Adult plants were exposed to −5° C./darkness for 5 hours followed by a one week recovery period under normal conditions (+22° C./LD). The number of plants surviving the freeze-shock exposure was determined after the recovery period. Experiments were performed in triplicate.

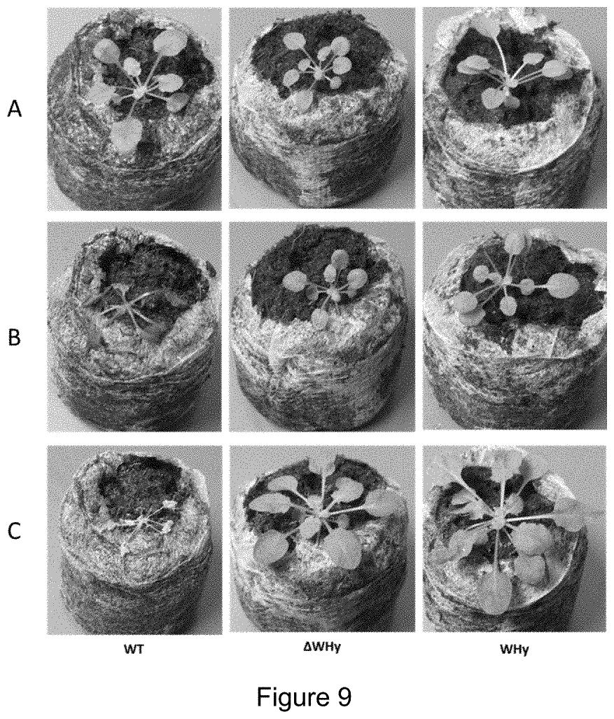

WT, ΔWHy and WHy Arabidopsis thaliana plants were grown for 4 weeks under standard conditions and then exposed to freezing (-5° C.) for 5 hours in darkness. FIG. 9 shows individual plants both before and directly after freeze treatment, and after a recovery period of 1 week under standard conditions. For freeze experiments, 5 adult plants were used and experiments were performed in triplicate. While WT plants showed significant freeze-damage immediately after freezing, all transgenic line plants showed obvious tolerance to freezing (FIG. 9), as indicated by continued growth during the 7 day recovery period.

Effect of Salinity on Transgenic Arabidopsis thaliana Seed Germination

Salt stress effects on seed germination were measured using different concentrations of NaCl (0, 50, 100, 150, 200 mM) in MS basal media plates. After surface sterilization, 10 seeds each of the WT and transgenic Arabidopsis thaliana lines were germinated on MS basal media in a controlled culture room at +22° C. under LD condition. After 2-3 weeks, the number of survivors/seedlings was determined. Experiments were performed in triplicate.

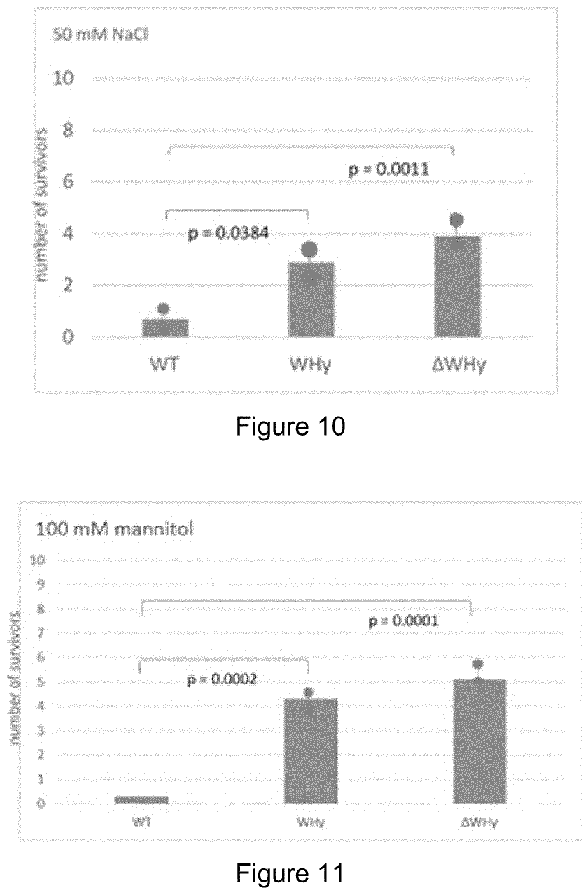

Quantitative analysis of Arabidopsis thaliana seeds germinated on media containing different concentrations of NaCl demonstrated a statistically significant higher tolerance of the recombinants towards salinity. All lines showed equal germination rates under standard growth conditions (data not shown) but a statistically significant (p=<0.05) higher number of survivors were observed for the transgenic lines when germinated on 50 mM sodium chloride in media petri dishes (FIG. 10). All plant lines (WT and recombinant) showed lethal sensitivity towards sodium chloride concentrations of 100 mM and above (Table 2).

Effect of Drought Stress on Transgenic Arabidopsis thaliana Seed Germination

Drought-tolerance effects on seed germination were measured using different concentrations of mannitol (0, 100, 200, 300 mM) in MS basal media plates. After surface sterilization, 10 seeds each of the WT and transgenic Arabidopsis thaliana lines were germinated on MS basal media in a controlled culture room at +22° C. under LD condition. After 2-3 weeks, the number of survivors/seedlings was determined. Experiments were performed in triplicate.

The phenotypic and physiological changes in WT and recombinant lines (WHy and ΔWHy) of Arabidopsis thaliana under water-stress were evaluated. Both WT and transgenic lines germinated well under standard growth conditions (data not shown). WT seeds showed poor germination when grown with 100 mM mannitol (FIG. 11) while both recombinant lines were found to be tolerant to mannitol concentrations of 200 mM (FIG. 11). Percent germination, for groups of 10 seeds, showed quantitative evidence of the higher resistance of recombinant seeds under drought conditions (Table 2).

TABLE 2
Germination of seeds under salinity/drought conditions in the MS basal media

| | Salinity | | | Drought | | |
|---|---|---|---|---|---|---|
| | Number of survivors | | | Number of survivors | | |
| | 0 mM | 50 mM | 100 mM | 0 mM | 100 mM | 200 mM |
| Line | NaCl | NaCl | NaCl | mannitol | mannitol | mannitol |
| WT | 7.0 + 1.0 | 0.7 + 1.2 | 0 + 0 | 6.7 + 1.2 | 0.3 + 0.6 | 0 + 0 |
| WHy 1 | 5.7 + 1.5 | 1.0 + 2.0 | 0 + 0 | 5.7 + 2.3 | 5.0 + 1.7 | 0.3 + 0.6 |
| WHy 2 | 6.0 + 1.0 | 4.3 + 1.2 | 0 + 0 | 5.3 + 1.2 | 3.7 + 0.6 | 0 + 0 |
| WHy 3 | 7.7 + 0.6 | 3.0 + 1.0 | 0 + 0 | 4.7 + 0.6 | 4.3 + 0.6 | 1.7 + 1.5 |
| ΔWHy 4 | 8.0 + 1.0 | 4.3 + 0.6 | 0 + 0 | 6.0 + 2.0 | 5.3 + 1.5 | 1.7 + 1.2 |
| ΔWHy 5 | 5.7 + 0.6 | 2.7 + 0.6 | 0 + 0 | 6.7 + 1.5 | 5.7 + 0.6 | 2.7 + 0.6 |
| ΔWHy 6 | 7.0 + 0 | 4.7 + 0.6 | 0 + 0 | 4.3 + 1.2 | 4.7 + 0.6 | 2.0 + 1.0 |

Effect of Long-Term Drought in WHy Plant Survival and Growth

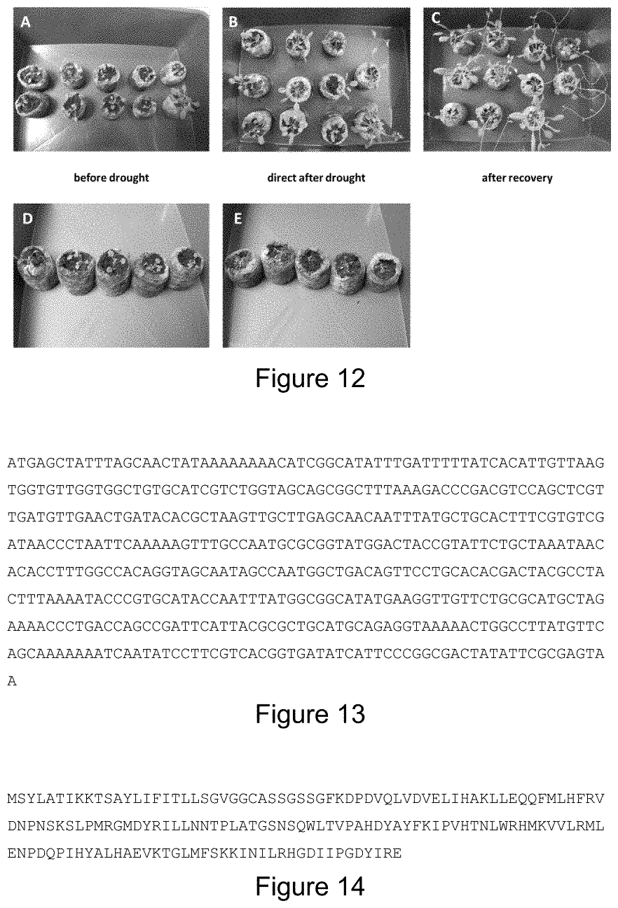

Five Arabidopsis thaliana plants each of WT, ΔWHy and WHy lines were grown for 3 weeks under normal conditions and exposed to drought (no water addition) for 1 week. Pictures of plants were taken before and directly after drought treatment, and after a recovery period of 1 week again under standard conditions. None of the WT plants survived the drought period (FIG. 12, Panel E), whereas all plants of the transgenic lines showed apparent tolerance to water-stress (FIG. 12, Panels B, C). Interestingly, it appeared that the drought period favored plant development and triggered early flowering (FIG. 12, Panel C: non-stressed controls not shown). In the present invention it was noted that under standard growth conditions, the inventors observed a typical time from germination to flowering of about 6-7 weeks (c.f. <5 weeks for the experimental plants shown).

### EXAMPLE 4

WHy Gene Expression in Different Tissues of Adult Arabidopsis thaliana Plants

Three Arabidopsis thaliana plants each of WT, ΔWHy and WHy lines were grown for 4 weeks under normal conditions (as described in Example 1 above). Roots, leaves and stem as well as untreated seeds of all lines were harvested and quick-frozen in liquid nitrogen. RNA was extracted using the NucleoSpin RNA Plant kit (Macherey-Nagel, Germany). RT-PCRs were conducted and generated cDNA was used in standard PCR using ΔWHy primers for all lines as described in Examples 1 and 2. PCR products were investigated via gel electrophoresis.

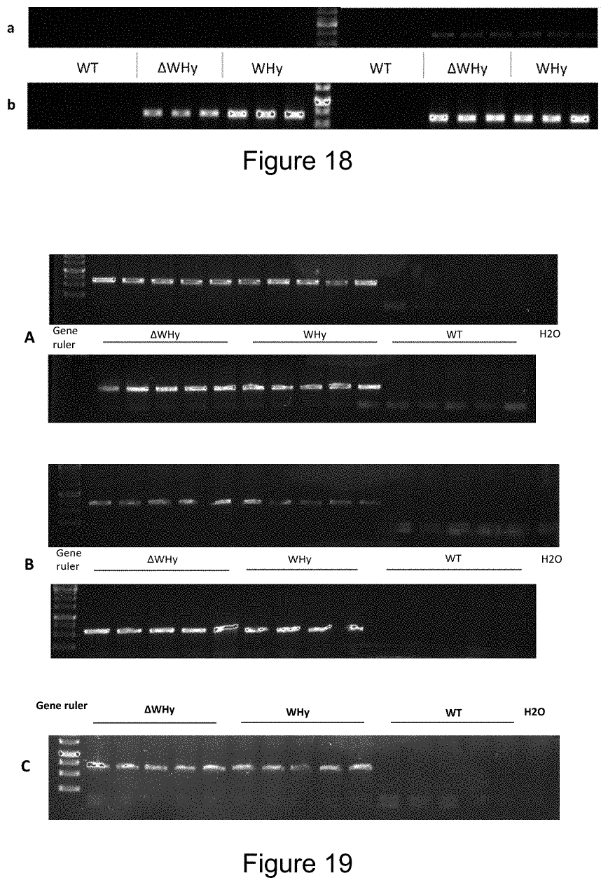

No WHy gene expression was found in untreated Arabidopsis thaliana seeds, while roots showed low expression compared to strong WHy expression in leaves and stems in all transgenic plants (FIG. 18). No WHy expression was detected in any WT tissue, as expected.

### EXAMPLE 5

### WHy Gene Expression During Abiotic Stress

Leaf material from five Arabidopsis thaliana plants each (WHy, ΔWHy and WT) was taken before and after freeze or drought shock and after seedling development under long periods of cold (+4° C.) conditions (detailed stress conditions as described in Example 3 above) and quick-frozen in liquid nitrogen. RNA was extracted and RT-PCR followed by normal ΔWHy-PCR were performed as described in Examples 1 and 2. PCR products were visualized via gel electrophoresis.

Before and directly after stress conditions a stabile WHy gene expression could be observed in both transgenic but in WT lines (FIG. 19).

### EXAMPLE 6

Generation of Transgenic Nicotiana benthamiana Plants

Sub-Cloning of ΔWHy

A codon-optimized DNA sequence for expression in Nicotiana benthamiana (SEQ ID NO:10) of the truncated variant of the WHy protein, lacking the predicted signal peptide and including a 6× His Tag (HHHHHH; SEQ ID NO:4) at the C-terminal domain of the protein (SEQ ID NO:2, FIG. 14), was also synthesised. The sequence of the Nicotiana benthamiana codon-optimized WHy gene encoding the truncated variant was amplified using the WHy forward primer (SEQ ID NO:8) designed to introduce a Kozak sequence (SEQ ID NO:11) and WHy reverse primer (SEQ ID NO:9), as described in Example 1. The Nicotiana benthamiana codon-optimised sequence further comprises an AfI III restriction site (ACATGT) at the 5′ end and a SaI I restriction site (GTCGAC) at the 3′ end.

The codon-optimised sequence was cloned in the pRIC and pTRAc vectors (Regnard et al), both leading to expression in the cytoplasm, for WHy protein expression in Nicotiana benthamiana. The codon-optimised sequence was also cloned into the pTRA-ERH vector for targeting WHy protein expression to the endoplasmic reticulum and the pTAkc-rbcsi-CTP vector for targeting WHy protein expression to the chloroplasts. All the vectors were provided by Prof Ed Rybicki of the University of Cape Town.

Transformation of Nicotiana benthamiana

Agrobacterium-mediated transformation of Nicotiana benthamiana was performed by vacuum infiltration and the WHy protein was transiently expressed in Nicotiana benthamiana.

### REFERENCES

Anderson D., et al (2015) A novel bacterial Water Hypersensitivity-like protein shows in vivo protection against cold and freeze damage. FEMS Microbiology Letters 362: fnv110

Laing W and Christeller J (2004) Extraction of Proteins from Plant Tissues. Current Protocols in Protein Science 4.7.1-4.7.7

Regnard G L, Halley-Stott R P, Tanzer F L, Hitzeroth I I, Rybicki E P (2010) High Level Protein Expression in Plants Through the Use of a Novel Autonomously Replicating Geminivirus Shuttle Vector. Plant Biotechnology Journal 8 pp. 38-46

Xin Z and Browse J (1998) Eskimol mutants of Arabidopsis are constitutively freezing-tolerant. Proceedings of the National Academic of Sciences, USA 95: 7799-7804

Zhang X, Henriques R, Lin S S, Niu Q W, Chua N H (2006). Agrobacterium-mediated transformation of Arabidopsis thaliana using the floral dip method. Nature Protocols 1: 2.

1. A transgenic plant having increased tolerance to an abiotic stress, wherein the transgenic plant is transformed with a nucleic acid sequence encoding a WHy protein comprising SEQ ID NO:2.

2. The transgenic plant of claim 1, wherein the nucleic acid sequence encoding the WHy protein is operably linked to a promoter.

3. The transgenic plant of claim 1, wherein the abiotic stress is selected from the group consisting of freeze stress, cold stress, heat stress, salt stress, drought stress, osmotic stress, water stress, oxidative stress, and ionic stress.

4. The transgenic plant of claim 1, wherein the increased tolerance to the abiotic stress is selected from the group consisting of increased germination efficiency, increased seedling survival, longer root length, faster plant development, increased seedling wet weight, increased plant growth, and early flowering.

5. The transgenic plant of claim 1, wherein the transgenic plant is stably transformed with the nucleic acid encoding the WHy protein.

6. The transgenic plant of claim 1, wherein the nucleic acid sequence is a synthetic codon-optimised sequence for expression in the plant.

7. A method for obtaining a transgenic plant having increased tolerance to an abiotic stress, the method comprising transforming a plant with a nucleic acid sequence encoding a WHy protein comprising SEQ ID NO:2.

8. The method of claim 7, wherein the method further comprises exposing the transgenic plant to an abiotic stress and selecting at least one plant having increased tolerance to the abiotic stress.

9. The method of claim 7, wherein the nucleic acid sequence encoding the WHy protein is operably linked to a promoter.

10. The method of claim 7, wherein the abiotic stress is selected from the group consisting of freeze stress, cold stress, heat stress, salt stress, drought stress, osmotic stress, water stress, oxidative stress, and ionic stress.

11. The method of claim 7, wherein the increased tolerance to the abiotic stress is selected from the group consisting of increased germination efficiency, increased seedling survival, longer root length, faster plant development, increased seedling wet weight, increased plant growth, and early flowering.

12. The method of claim 7, wherein the transgenic plant is stably transformed with the nucleic acid encoding the WHy protein.

13. The method of claim 7, wherein the nucleic acid sequence is a synthetic codon-optimised sequence for expression in the plant.

14. Vegetative tissue from the transgenic plant of claim1.

15. A transgenic seed containing a nucleic acid sequence encoding a WHy protein comprising SEQ ID NO:2.


SEQUENCE LISTING (11 sequences)

SEQ ID NO: 1 - DNA, 497 bp, Unknown
  WHy gene metagenomic library isolate nucleotide sequence
atgagctatttagcaactataaaaaaaacatcggcatatttgatttttatcacattgttaagtggtgttggtggctgtgcatcgtctggtagcagcggctttaaagacccgacgtccagctcgttgatgttgaactgatacacgctaagttgcttgagcaacaatttatgctgcactttcgtgtcgataaccctaattcaaaaagtttgccaatgcgcggtatggactaccgtattctgctaaataacacacctttggccacaggtagcaatagccaatggctgacagttcctgcacacgactacgcctactttaaaatacccgtgcataccaatttatggcggcatatgaaggttgttctgcgcatgctagaaaaccctgaccagccgattcattacgcgctgcatgcagaggtaaaaactggccttatgttcagcaaaaaaatcaatatccttcgtcacggtgatatcattcccggcgactatattcgcgagtaa

SEQ ID NO: 2 - protein, 165 aa, Unknown
  WHy gene metagenomic library isolate amino acid sequence
MSYLATIKKTSAYLIFITLLSGVGGCASSGSSGFKDPDVQLVDVELIHAKLLEQQFMLHFRVDNPNSKSLPMRGMDYRILLNNTPLATGSNSQWLTVPAHDYAYFKIPVHTNLWRHMKVVLRMLENPDQPIHYALHAEVKTGLMFSKKINILRHGDIIPGDYIRE

SEQ ID NO: 3 - DNA, 516 bp, Artificial sequence
  Arabidopsis codon-optimised WHy nucleotide sequence
atgtcatacttggctactatcaaaaagacctctgcttacctcatattcataaccttgctctcaggagtgggtggttgtgcttcatcaggatcttcaggttttaaggatcctgatgttcaactcgttgatgtggaattaatccatgctaaacttttggagcaacagtttatgcttcacttcagagtggataatcctaattctaagtctcttccaatgaggggtatggattacaggatactcctcaataacactccactcgcaacaggttcaaatagtcaatggttaaccgttcctgctcatgattatgcatactttaagataccagtgcatactaatctctggagacacatgaaagttgtgcttaggatgttggaaaaccctgatcagccaattcattacgctcttcacgcagaggttaaaacaggattgatgttctctaagaaaattaacatcttgagacacggagatattataccaggagattacatcagggagcaccaccatcatcaccattga

SEQ ID NO: 4 - protein, 6 aa, Artificial sequence
  Histidine tag
HHHHHH

SEQ ID NO: 5 - DNA, 447 bp, Artificial sequence
  Arabidopsis codon-optimised truncated WHy nucleotide sequence
atgggatgtgcttcatcaggatcttcaggttttaaggatcctgatgttcaactcgttgatgtggaattaatccatgctaaacttttggagcaacagtttatgcttcacttcagagtggataatcctaattctaagtctcttccaatgaggggtatggattacaggatactcctcaataacactccactcgcaacaggttcaaatagtcaatggttaaccgttcctgctcatgattatgcatactttaagataccagtgcatactaatctctggagacacatgaaagttgtgcttaggatgttggaaaaccctgatcagccaattcattacgctcttcacgcagaggttaaaacaggattgatgttctctaagaaaattaacatcttgagacacggagatattataccaggagattacatcagggagcaccaccatcatcaccattga

SEQ ID NO: 6 - protein, 148 aa, Artificial sequence
  Truncated WHy amino acid sequence
MGCASSGSSGFKDPDVQLVDVELIHAKLLEQQFMLHFRVDNPNSKSLPMRGMDYRILLNNTPLATGSNSQWLTVPAHDYAYFKIPVHTNLWRHMKVVLRMLENPDQPIHYALHAEVKTGLMFSKKINILRHGDIIPGDYIREHHHHHH

SEQ ID NO: 7 - DNA, 27 bp, Artificial sequence
  WHy forward primer nucleotide sequence
gccaccatgggatacttggctactatc

SEQ ID NO: 8 - DNA, 25 bp, Artificial sequence
  Truncated WHy forward primer nucleotide sequence
gccaccatgggatgtgcttcatcag

SEQ ID NO: 9 - DNA, 25 bp, Artificial sequence
  WHy reverse primer nucleotide sequence
tcaatggtgatgatggtggtgctcc

SEQ ID NO: 10 - DNA, 453 bp, Artificial sequence
  Nicotiana Benthamiana codon-optimised WHy nucleotide sequence
gccaccatgggatgtgcttcatcaggaagttcaggtttcaaagatccagatgtgcagttagttgatgtggagcttattcacgcaaagttattggagcaacagtttatgcttcacttcagagttgataaccctaactctaagtcacttccaatgaggggtatggattacaggatccttttgaataacactcctcttgctacaggatctaattcacaatggttgactgttcctgctcatgattacgcttacttcaagatcccagttcatacaaatttgtggagacatatgaaagttgttcttaggatgttggaaaatcctgatcagccaattcattacgctcttcatgctgaggttaaaactggtttgatgttcagtaagaagattaacattttgagacacggagatattattccaggagattacattagggagcaccaccatcatcaccattga

SEQ ID NO: 11 - DNA, 10 bp, Artificial sequence
  Kozak sequence
gccaccatgg